(19) United States
(12) Patent Application Publication
(10) Pub. No.: US 2015/0250413 A1
(43) Pub. Date: Sep. 10, 2015

(54) SYSTEMS AND APPARATUS FOR NEEDLE-PENETRATION PAIN MANAGEMENT

(71) Applicant: eldeeN, LLC, Cincinnati, OH (US)

(72) Inventors: Racquel Redwood, Cincinnati, OH (US); Joel Fladen, Centerville, OH (US)

(21) Appl. No.: 14/642,440

(22) Filed: Mar. 9, 2015

Related U.S. Application Data
(60) Provisional application No. 61/949,403, filed on Mar. 7, 2014.
(60) Provisional application No. 61/974,270, filed on Apr. 2, 2014.

(51) Int. Cl. A61B 5/15; A61B 19/02; A61B 5/151

(52) U.S. Cl. CPC A61B 5/150137; A61B 5/150839; A61B 5/150267; A61B 5/151; A61B 19/0262; A61B 5/150854; A61B 2503/06

(57) ABSTRACT

A needle-penetration pain management system for managing needle penetration pain can include a needle-penetration assembly including a needle, the needle having a proximal end and a distal end, an encasement apparatus including a housing, the housing having a front surface, a rear surface, and a base, where the housing at least partially defines an interior chamber for installing the needle-penetration assembly at least partially therein, a stimulation generator for producing stimuli, the stimulation generator being coupled with the housing, and an actuator in communication with the needle-penetration assembly for causing the needle to puncture a skin surface of a user in response to actuation.

### REFERENCE TO RELATED APPLICATIONS

This application claims priority to U.S. Provisional Patent Application No. 61/949,403, entitled SYSTEMS AND APPARATUS FOR NEEDLE-PENETRATION PAIN MANAGEMENT, filed Mar. 7, 2014 and U.S. Provisional Patent Application No. 61/974,270, entitled SYSTEMS AND APPARATUS FOR NEEDLE-PENETRATION PAIN MANAGEMENT, filed Apr. 2, 2014, each of which is hereby incorporated herein by reference in its entirety.

### TECHNICAL FIELD

Embodiments of the technologies described herein relate, in general, to needle sticking, scraping, and puncturing devices, and, in particular, to methods, devices, and apparatuses for pain management of needle sticking, scraping, and puncturing devices.

### BACKGROUND

Patients diagnosed with diabetes are typically required by their doctors to test their blood glucose levels. Oftentimes, such tests must be performed multiple times throughout the day. To do so, patients typically prick a finger or other part of their anatomy to obtain a blood sample, which is then analyzed by a blood glucose meter. This process is then repeated throughout the day. Non-compliance with a testing regimen may occur due to the pain and apprehension of performing a finger prick with a lancing device. This is especially true in pediatric patients.

### BRIEF DESCRIPTION OF THE DRAWINGS

The present disclosure will be more readily understood from a detailed description of some example embodiments taken in conjunction with the following figures in which:

FIG. 1 depicts a perspective view of an exemplary encasement apparatus for a needle-penetration assembly according to one embodiment;

FIG. 2 is a front view of the exemplary encasement apparatus depicted in FIG. 1;

FIG. 3 is a bottom view of the encasement apparatus depicted in FIG. 1;

FIG. 4 is a right side view of the encasement apparatus depicted in FIG. 1;

FIG. 5 is an angled perspective view illustrating the internal layout of the encasement apparatus depicted in FIG. 1;

FIG. 6 depicts the separated components of an encasement device according to one embodiment;

FIGS. 7A-7F illustrate at least one embodiment of a method for operating an encasement apparatus;

FIG. 8 depicts a blood glucose monitor;

FIG. 9 depicts a needle-penetration assembly according to one embodiment;

FIGS. 10A-10B depict an encasement apparatus according to another embodiment;

FIG. 11 depicts an encasement apparatus according to an another embodiment;

FIG. 12 depicts an alternative encasement apparatus according to yet another embodiment;

FIG. 13 depicts a needle-penetration assembly according to one embodiment;

FIG. 14 depicts a needle-penetration assembly according to another embodiment; and

FIG. 15 depicts an alternate version of a needle penetration assembly and encasement according to one embodiment.

FIGS. 16 and 17 depict a simplified flow diagram of at least one embodiment of a method for operating an encasement apparatus.

### SUMMARY

A needle-penetration pain management system for managing needle penetration pain can include a needle-penetration assembly including a needle, the needle having a proximal end and a distal end, an encasement apparatus including a housing, the housing having a front surface, a rear surface, and a base, where the housing at least partially defines an interior chamber for installing the needle-penetration assembly at least partially therein, a stimulation generator for producing stimuli, the stimulation generator being coupled with the housing, and an actuator in communication with the needle-penetration assembly for causing the needle to puncture a skin surface of a user in response to actuation.

A needle-penetration pain management system for managing needle penetration pain including a needle-penetration assembly including a needle, the needle having a proximal end and a distal end, an encasement apparatus including a housing resembling an animal, the housing having a front surface, a rear surface, and a base, where the housing at least partially defines an interior chamber and an aperture in the base for installing the needle-penetration assembly at least partially therein, a first chamber, defined by the housing, for the storage of materials, a first stimulation generator for producing a vibrational stimulus, the first stimulation generator being coupled with the housing, where the first stimulation generator is selectively adjustable between a plurality of modes of vibrational stimuli, a second stimulation generator for producing a visual stimulus, a third stimulation generator for producing an audible stimulus, a transmitter, a receiver, and a controller associated with the housing, where the transmitter and the receiver are configured to communicate with a remote device over a network, and an actuator in communication with the needle-penetration assembly for causing the needle to puncture a skin surface of a user in response to actuation.

A needle-penetration pain management system including a needle assembly means, and an encasement assembly means having, a housing means, a stimulation means, and an actuator means.

### DETAILED DESCRIPTION

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment”, or “in an embodiment” in places throughout the specification are not necessarily all referring to the same embodiment. Furthermore, the particular features, structures or characteristics may be combined in any suitable manner in one or more embodiments.

Described herein are example embodiments of apparatuses, systems, and methods for pain management of needle-penetration devices. In one example embodiment, the pain management can be provided through an assistive integrated pain management system. The assistive integrated pain management system can use stimuli such as, for example, temperature, auditory, psychological, tactile, or combinations of the forgoing to reduce, or eliminate, the perception of needle-penetration pain. As will be appreciated, the stimuli useful to the assistive integrated pain management system is not limited to the foregoing and can additionally include visual, taste, and/or electric stimuli. Stimuli can be provided through various mechanisms, including, for example, chemical reactions, substrates, gels, conductive substances, pain-relieving ointments, and nerve receptor agents such as Icy-Hot®. In some embodiments, the assistive integrated pain management can include an encasement apparatus to provide the stimuli. Additionally, in some embodiments, the encasement apparatus can represent identifiable objects, organisms, or characters.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Example embodiments described herein can mitigate and distract the pain and discomfort of needle-penetration devices. Without being bound to the theory, the embodiments can utilize principles derived from the Gates Theory of Pain to minimize the perception of pain. The Gates Theory of Pain suggests the stimulation of nerves adjacent to a needle penetration point can be used to eliminate, mask, or distract the associated pain because the stimulation activates similar parts of the brain as the pain and overshadows the perception of pain. Increasing the quantity and quality of sensory nerves stimulated simultaneous to, and adjacent with, the painful nerve stimulation, can further lessen the pain perceived. Example embodiments disclosed can provide a plurality of stimuli to reduce the perceived sensation of pain from a needle penetration device.

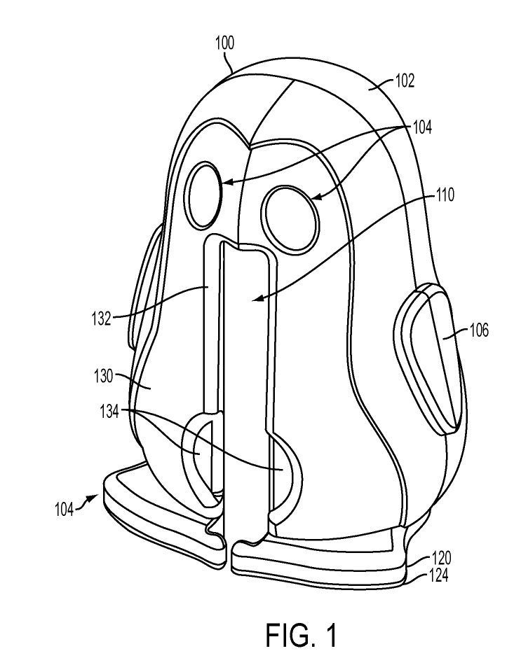

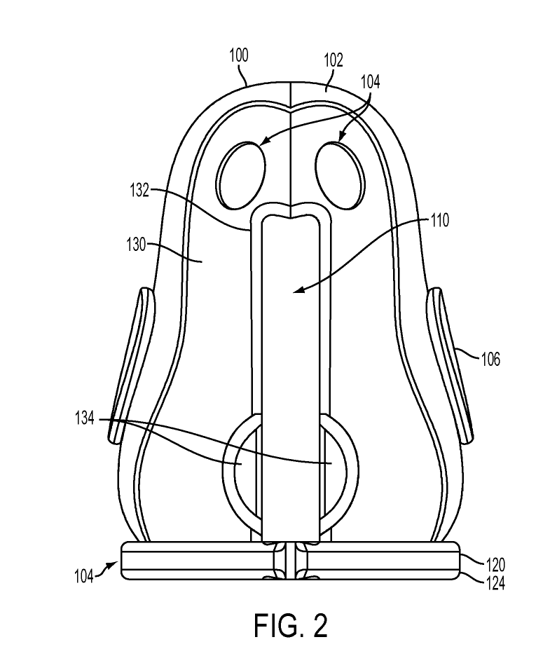

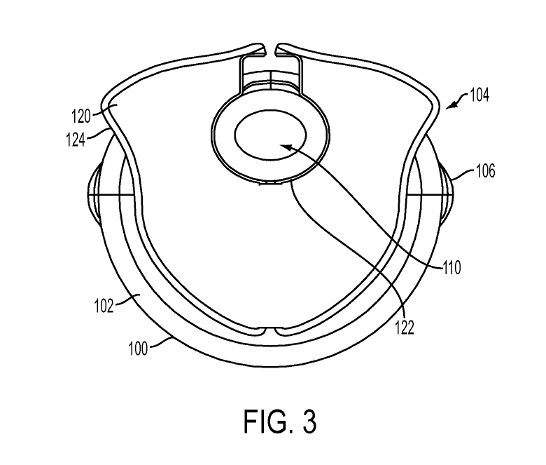

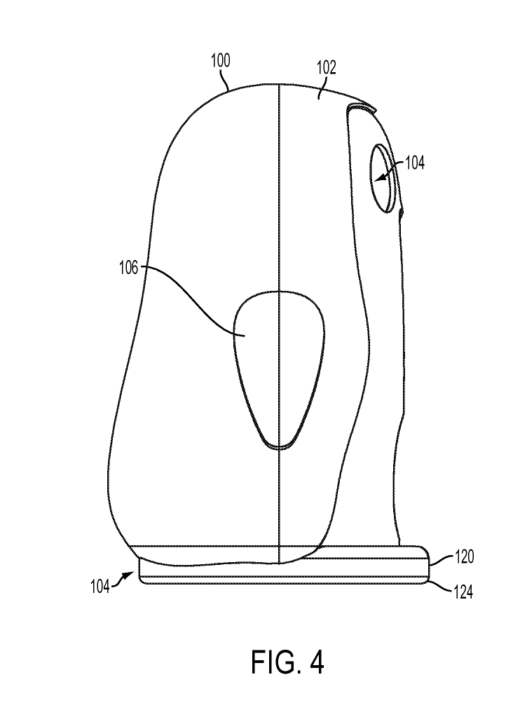

Referring now to FIGS. 1-6, the pain management system can be an assistive integrated pain management system that includes an encasement apparatus 100. Generally, the encasement apparatus 100 includes a housing 102, one or more stimulation generators 104, and an actuator 106. The housing 102 can define an interior chamber 110 configured to receive a needle-penetration assembly 150. In some embodiments, the housing 102 and/or the interior chamber 110 can be adjustable to facilitate secure attachment of a multitude of needle-penetration assemblies 150 having different shapes, sizes, materials, mechanisms, components, applications and/or configurations. In some embodiments, a front wall 130 of the housing 102 can include an aperture 132 in communication with the interior chamber 110. Additionally, the front wall 130 of the housing 102 can further include recessed portions 134 or compartments. In such embodiments, the aperture 132 and/or the recessed portions 134 can be configured to provide access to the needle-penetration assembly 150 without disrupting the psychological stimulus provided by the exterior shape of the encasement apparatus 100.

In some embodiments, the encasement apparatus 100 includes a base 120 for supporting the housing 102 and/or assisting an operator in correctly aligning and placing the encasement apparatus 100 on a patient or user. The base 120 may be separately attached to the housing 102 or it may form an integral part thereof. Additionally, the base 120 can include a textured surface 124 as discussed in more detail below. In some embodiments, the base 120 can define or partially define an aperture 122 in communication with the interior chamber 110. In such embodiments, the aperture 122 can be configured to facilitate mounting or installation of needle-penetration assembly 150 into the interior chamber 110 of the encasement apparatus 100.

Additionally, in some embodiments, the encasement apparatus 100 can further include attachment mechanisms to assist in the use of the needle-penetration assembly 150. Example attachment mechanisms can include harnesses, straps, adhesives, or the use of pressure such as in a vacuum or suction-type system. The attachment mechanisms can assist an operator in correctly aligning and placing the apparatus 100 on the patient or user.

The encasement apparatus 100 can be constructed out of any suitable material. In some embodiments, this material can be selected to present a soft feel to users (e.g., operators, patients, etc.) to eliminate irritation or itching in users. It should also be appreciated that materials that provide a suitable level of resistance, or that have a textured surface can also be used independently or in combination with other materials. Additionally or alternatively, the material can also be selected for antiseptic qualities, the ability of the material to be sterilized, and the biocompatibility of the material. For example, nitrile materials can be selected to avoid latex allergies. In some embodiments, the material can be a reprocessed material. Additionally, in some embodiments, materials capable of autoclave sterilization may be selected. It should be appreciated that the components (e.g., the housing 102, actuator 106, base 120, etc.) of the encasement apparatus 100 may each be constructed out of substantially similar materials. In some embodiments, however, one or more of the components of the encasement apparatus 100 may be constructed out of different materials than the remaining components.

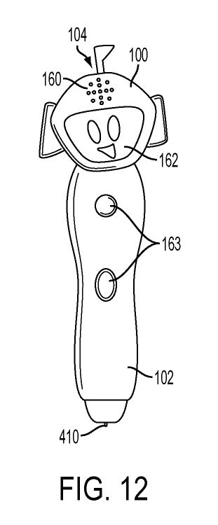

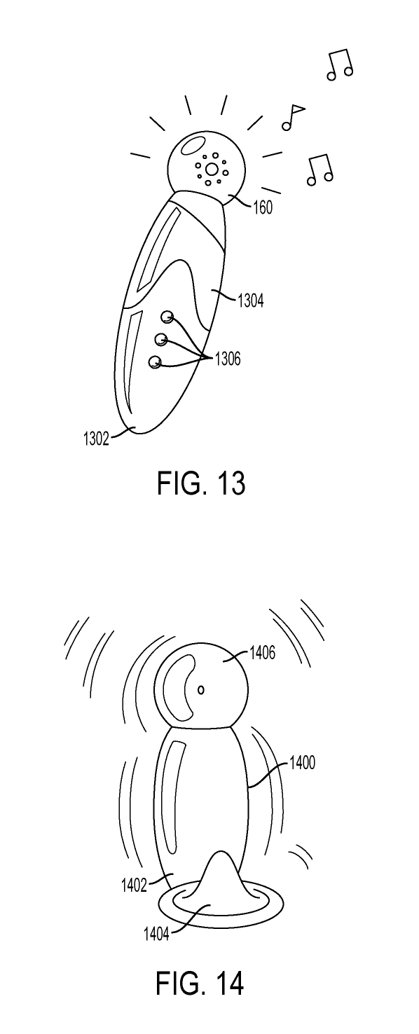

The stimulation generators 104 can be integrated into the encasement apparatus 100 and can be configured to produce various forms of auditory, visual, somatosensory, gustatory, and electric stimuli to reduce, or eliminate, the perception of needle-penetration pain. For example, in some embodiments, one or more of the simulation generators 104 can include, or may otherwise be embodied as, a speaker (e.g., the speakers 160 illustratively shown in FIGS. 12 and 13) configured to generate or reproduce various sounds as audible stimuli. Such sounds may be embodied as one or more beeps, buzzes, rings, tones, notes, chords, music, and any other type of sound capable of being generate and/or reproduced by a speaker. The speaker can generate and/or reproduce the sounds in a variety of customizable pitches and tones at different volumes and for variable durations and frequencies. Additionally or alternatively, in some embodiments, one or more of the stimulation generators 104 may be embodied as a mechanical stimulation generator 104. For example, in such embodiments, a stimulation generator 104 may be embodied as a mechanical rattler, clicker, and/or shutter configured to generate corresponding sounds as the audible stimuli.

Additionally or alternatively, in some embodiments, one or more of the stimulation generators 104 can include, or otherwise may be embodied as, a light source or a display configured to emit light or render content as visible stimuli. In such embodiments, the light source or display can emit light in a variety of customizable colors at varying intensities, durations, and frequencies. In some embodiments, one or more of the stimulation generators 104 may be embodied as a light-emitting diode (LED). Additionally or alternatively, in some embodiments, one or more of the stimulation generators 104 may be embodied as chemical stimulation generator 104. For example, in such embodiments, a stimulation generator 104 may include temperature sensitive liquid crystals configured to convey the visual stimuli. In another example, one or more of the stimulation generators 104 may be embodied as a display (e.g., the display 162 illustratively shown in FIG. 12) configured to convey visual stimuli to the user.

In some embodiments, one or more of the stimulation generators 104 can generate somatosensory and/or gustatory stimuli. For example, a stimulation generator 104 may include, or otherwise may be embodied as, symmetrical or asymmetrical motors configured to generate vibrations or provide tactile feedback to a patient or an operator of the encasement apparatus 100. One or more stimulation generators 104 may also include a textured surface. For example, in some embodiments such as the one illustratively shown in FIG. 1, the base 120 may include a textured surface 124 configured to be sensed by a patient or user when the encasement apparatus 100 is placed on the patient or user. In other examples, the base 120 may include, or otherwise may be embodied as a heat generator configured to provide temperature stimulation directly adjacent to the needle-penetration assembly 150 when the encasement apparatus 100 is placed on the patient and/or in response to actuation of, or interaction with, the actuator 106. In such examples, the base 120 can be considered to be a stimulation generator 104.

It should be appreciated that the stimulation generator(s) 104 may include, or otherwise may be embodied as, any other type of stimulation generator 104 configured to generate auditory, visual, somatosensory, gustatory, or other stimuli. For example, one or more of the stimulation generator(s) 104 may include, or may otherwise be embodied as, one or more chemicals, substrates, gels, conductive substances or materials, pain-relieving ointments, and nerve receptor agents such as Icy-Hot® capable of producing stimuli. Additionally, in some embodiments, one or more of the stimulation generators 104 may generate corresponding stimuli responsive to use of the encasement apparatus 100 and/or the needle-penetration assembly 150. For example, in some embodiments, one or more of the stimulation generators 104 may generate corresponding stimuli in response to actuation of, or interaction with, the actuator 106. In other embodiments, one or more of the stimulation generators 104 may generate corresponding stimuli independent of use of the encasement apparatus 100 and/or needle-penetration assembly 150. In either case, it should be appreciated that the stimulation generators 104 can provide a distracting stimulus effect that can further mask or override the pain perception of a needle of the needle-penetration assembly 150.

The encasement apparatus 100 can cooperate, incorporate, integrate, or attach with the needle-penetration assembly 150 to control operation of a needle of the needle-penetration assembly 150. For example, in some embodiments, the actuator 106 can actuate or cause the needle of the needle-penetration assembly 150 to puncture the skin of a user. In such embodiments, the actuator 106 can be mechanically or electrically coupled to the needle-penetration assembly 150 such that interaction with the actuator 106 (e.g., movement of the actuator 106, depressing the actuator 106, etc.) by a user causes the needle of the needle-penetration assembly 150 to puncture the skin of the user.

Additionally, in some embodiments, components of the encasement apparatus 100 can allow control and adjustment of operational parameters of the needle-penetration assembly 150 such as the needle penetration depth and penetration velocity of the needle. Such components may be mounted on and/or incorporated into the encasement apparatus 100 to facilitate such control and adjustment. Suitable components to control and adjust the operational parameters of the needle-penetration assembly 150 can include, among others, springs, clamps, gears, adhesives, levers, wedges, hinges, swings, locks, tape, snaps, screws, grooves, slides, knobs, pulleys, straps, and Velcro®. It should be appreciated that such components can also be used to secure and/or mount the needle-penetration assembly 150 into the interior chamber 110.

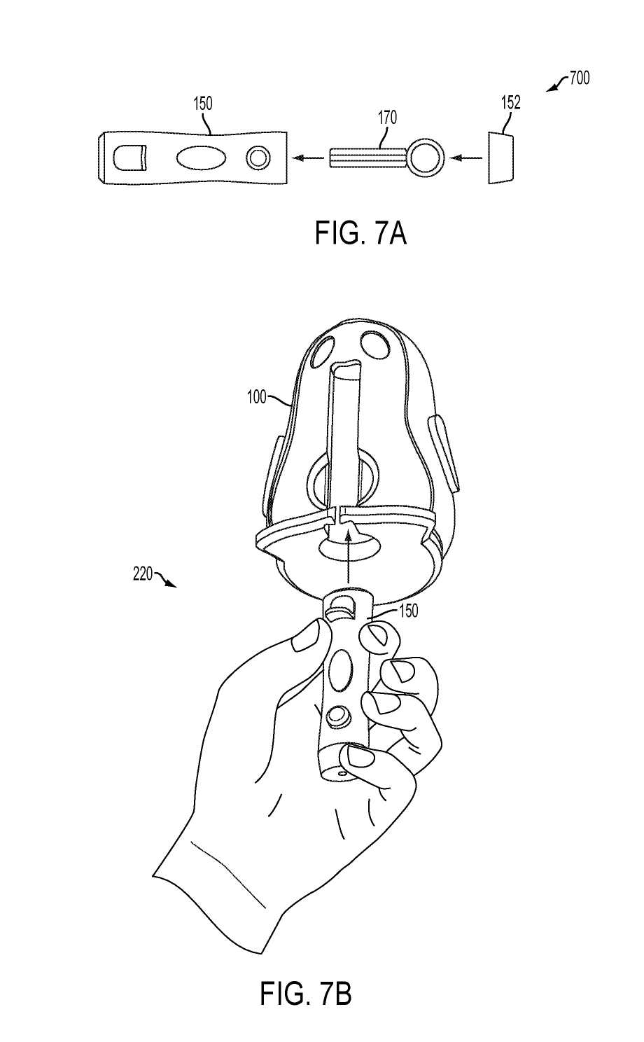

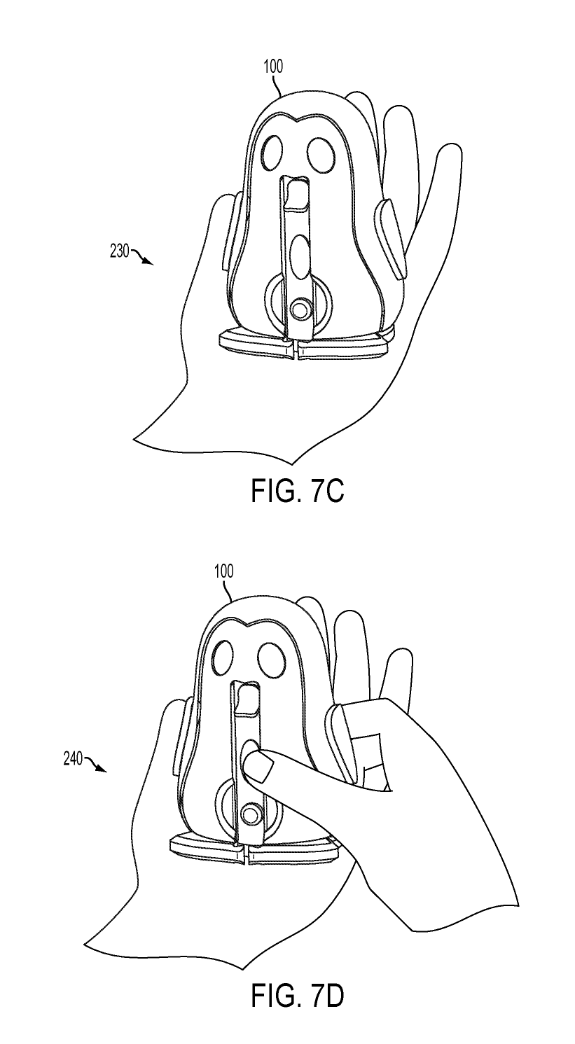

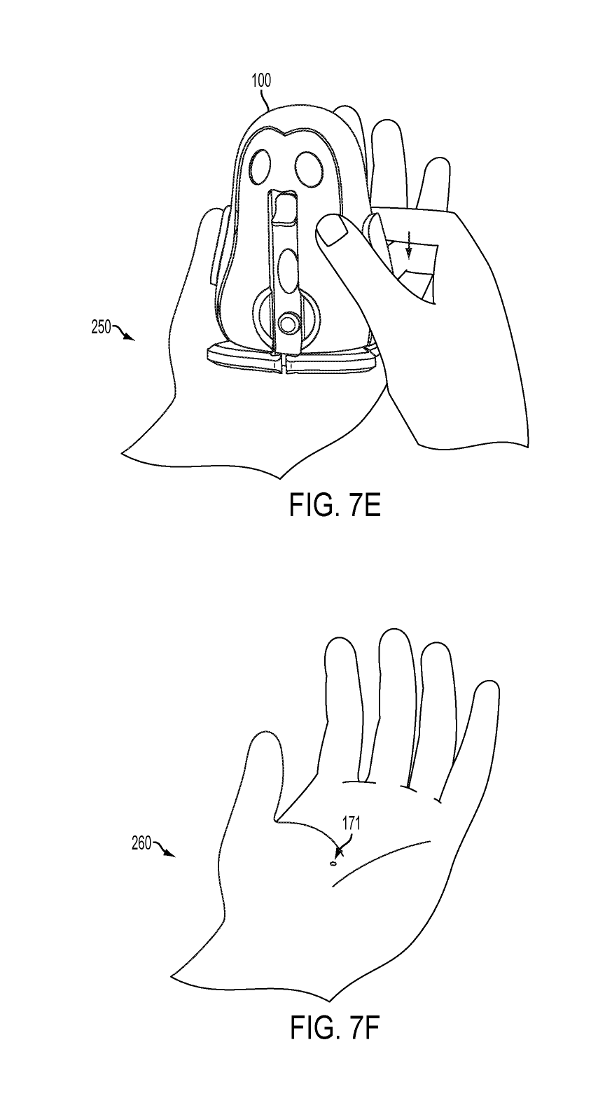

The encasement apparatus 100 can itself provide visual and psychological stimuli through an exterior shape of the housing 102. To do so, the exterior shape of the housing 102 of the encasement apparatus 100 can resemble or represent identifiable objects, organisms, or characters. For example, in some embodiments such as the one illustratively shown in FIGS. 1-6, the exterior shape of the housing 102 can be shaped to resemble a penguin or other animal. In such embodiments, the stimulation generators 104, actuator 106, base 120, and other components of the encasement apparatus 100 can each be shaped and located to resemble a different anatomical feature or characteristic of a penguin (or other animal). For example, as illustratively shown in FIG. 1, one or more stimulation generators 104 can be shaped and placed to resemble eyes of a penguin, other stimulation generators 104 can be shaped and placed to resemble feet of a penguin, the actuator 106 can be shaped and placed to resemble a wing of a penguin, and the front wall 130 of the housing 102 can be shaped and placed to resemble a stomach of a penguin. The encasement apparatus 100 illustratively shown in FIG. 1 can also include other features that resemble anatomical features or characteristics of a penguin. For example, in some embodiments, a portion of the aperture 132 of the front wall 130 may be configured to resemble a mouth of a penguin when a needle-penetration assembly 150 including a “beak” (e.g., the beak illustratively shown in FIGS. 7B-7E) is installed or mounted into the interior chamber 110 of the encasement apparatus 100.

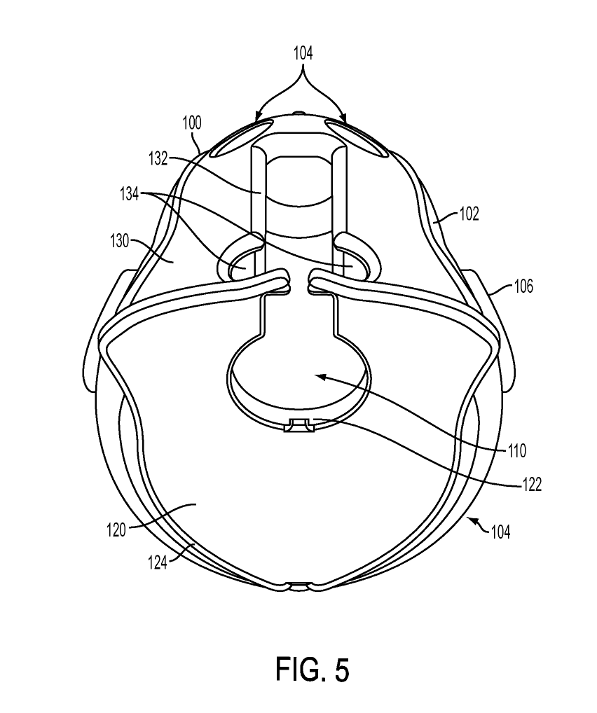

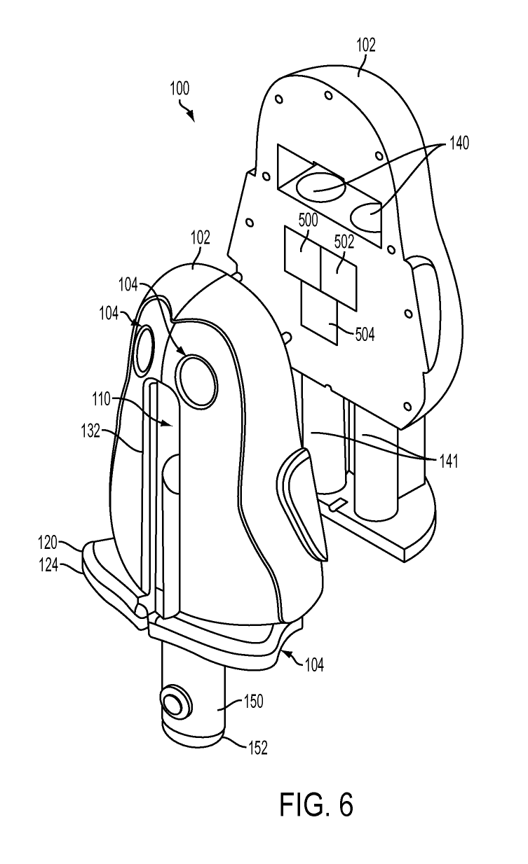

In embodiments using electrical components, the encasement apparatus 100 can include a power source. As illustratively shown in FIG. 6, in some embodiments, the power source can be one or more disposable or permanent rechargeable batteries 141. In such embodiments, the housing 102 of the encasement apparatus 100 may define one or more additional interior chambers or battery slots 140. The battery slots 140 can be configured to receive the one or more disposable or permanent rechargeable batteries 141. It should be appreciated that the power source may include, or may otherwise be embodied as, any other type of power source configured to provide power to one or more electrical components of the encasement apparatus 100. For example, in some embodiments, the power source may include, or may be embodied as, a solar rechargeable system, a hand crank, or electronic pack. In some embodiments, the power source (e.g., the disposable or permanent rechargeable batteries 141, solar rechargeable system, hand crank, electronic pack, etc.) can be removed as necessary. For example, in embodiments in which the encasement apparatus 100 is to be subjected to autoclave sterilization, the power source may be removed to prevent damage.

Referring to FIG. 6, the encasement apparatus can include a transmitter 500, a receiver 502, and a processor or controller 504. Example embodiments described herein can allow for the encasement apparatus 100 and associated systems to be operated using mobile devices or other remote devices. The controller 504 can execute software for operating the encasement apparatus 100, communicating with a remote network via the transmitter 500 and receiver 502, providing pre-programmed stimuli packages, and the like. The computer system can run on any suitable computing system, such as a dedicated server, a user computer or server, multiple computers, a collection of networked computers, a cloud-based computer system, a web-based computer system, or from a storage device, for example. One or multiple processing units, such as central processing units and/or graphics processing units, may perform instructions stored in memory to execute the processes described herein.

A computer system in accordance with the present disclosure can be accessed via any suitable technique, such as a web-browser such as SAFARI, OPERA, GOOGLE CHROME, INTERNET EXPLORER, or the like executing on a client device. In some embodiments, the systems and methods described herein can be a web-based application or a stand-alone executable. Additionally, in some embodiments, the systems and methods described herein can integrate with various types of electronic medical records and the like. Any suitable client device can be used to access, or execute, the computing system, such as laptop computers, mobile devices, desktop computers, smart phones, tablet computers, and the like.

Systems and methods described herein may generally provide an interactive environment for users (e.g., users can interact with the encasement apparatus 100 on their smartphone) over a network, such as a local area network, to initiate, create, monitor, or otherwise utilize the encasement apparatus 100. User interaction with the controller 504 may take place in any of a variety of operational environments, such as a clinical setting or a home setting, with one or more users interacting with the system at a given time.

In some embodiments, the encasement apparatus 100 can be configured to aid the effectiveness of the needle-penetration assembly 150. For example, the encasement apparatus 100 can include protective mechanisms such as protective caps 152 and dampening elements to protect the needle-penetration assembly 150 against damage or accidental use. The protective caps 152 and dampening elements can also provide support to the needle-penetration assembly 150, maintain sterility, and aid in the adaptability of the needle-penetration assembly 150. Additionally, the encasement apparatus 100 can partially, or completely, conceal, mask, or disguise the needle-penetration assembly 150. As can be appreciated, such concealment, masking, or disguising can act as a psychological element to reduce, or distract from, the perception of pain.

FIGS. 7A-7E depict a method 700 of using the encasement apparatus 100 as part of a complete assistive pain management system for needle penetration. In the method 700, a needle-penetration assembly 150 can be provided in step 210. The needle-penetration assembly 150 can include a blood lancet 170 or other type of needle. In some embodiments, the needle-penetration assembly 150 can include the protective cap 152 to protect the needle-penetration assembly 150 against damage or accidental use. In some embodiments, the needle can be adjusted for a specific puncture depth, velocity, penetration duration, as well as for the frequency and amplitude of any generated vibration. At step 220, the needle-penetration assembly 150 is secured within the encasement apparatus 100. Continuing at step 230, the encasement apparatus 100 is ready for use at this point and can be positioned at a suitable location on the patient such as on a palm. It will be appreciated that the location can be selected to provide an optimal reduction in perceived pain. In contrast to traditional needle injection, stimuli can be activated at step 240 to mask the pain of needle penetration, which can occur at step 250. At step 260, the method 700 completes with removal of the encasement apparatus 100 after actuation of the needle-penetration assembly 150. As illustratively shown in FIG. 7F, actuation of the needle-penetration assembly 150 in step 260 causes the needle of the needle-penetration assembly 150 to puncture the skin of the user at a puncture point 171.

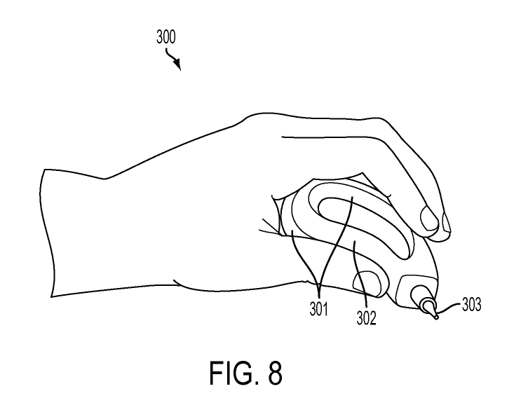

In some embodiments, the needle-penetration assembly 150 can be an independent lancing device system. Lancing systems can be useful, for example, for diabetic patients who need to consistently check blood glucose levels multiple times per day by collecting a drop of blood and measuring the glucose level on a blood glucose meter 300 such as the one illustratively shown in FIG. 8. The illustrative blood glucose meter 300 includes a body 301, a display, and a test strip 303, testing sensor, or testing chamber. The blood glucose meter 300 can be used in conjunction with the encasement apparatus 100. In some embodiments, an independent lancing system such as the needle-penetration assembly 150 may be installed and/or mounted into the interior chamber 110 of the housing 102 of the encasement apparatus 100 as discussed. In such embodiments, the blood glucose meter 300 can be used to check blood sugar levels with a diminished perception of pain. It should also be appreciated that in some embodiments, the needle-penetration assembly 150 can also be an integral lancing system with an integrated blood glucose monitor. In other embodiments, the needle-penetration assembly 150 can provide an injection or withdraw blood from a patient.

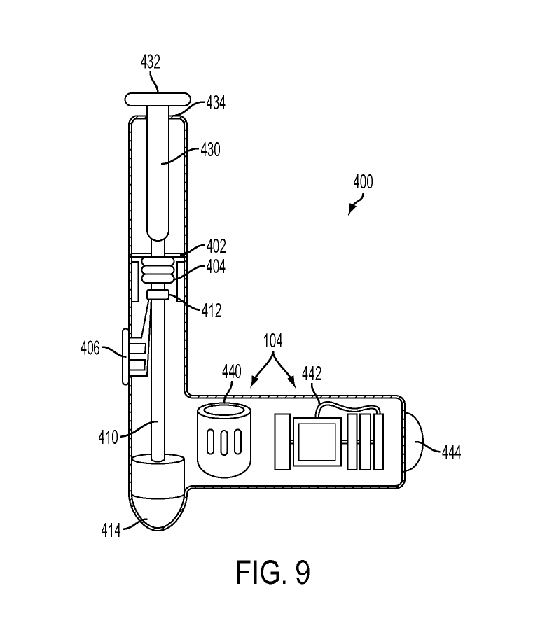

Referring now to FIG. 9, another exemplary needle-penetration assembly 400 is illustratively shown. The needle-penetration assembly 400 includes a plate 402, a spring 404, a button 406, and a needle 410. In some embodiments, actuation of or interaction with the button 406 causes a flange 412 (e.g., a proximal or medial flange) of the needle 410 to be released from a loaded position. In such embodiments, the elastic potential energy stored in the spring 404 is released causing the needle 410 to thrust forward, extend away from a protective cap 414, and puncture the skin of a user.

In some embodiments, the penetration depth of the needle 410 is adjustable. In such embodiments, the needle-penetration assembly 400 includes a penetration depth assembly 430. The penetration depth assembly 430 includes a proximal flange 432 configured to stop the forward thrust of the needle 410 when the proximal flange 432 contacts a top surface 434 of the needle-penetration assembly 400. In some embodiments, the penetration depth assembly 430 is adjustable relative to the needle 410. For example, in some embodiments, the penetration depth assembly 430 may include a threaded aperture configured to accept a threaded proximal end of the needle 410. In such embodiments, the needle-penetration depth can be adjusted by rotating the penetration depth assembly 430 clockwise or counter-clockwise relative to the needle 410. In doing so, the space between the proximal flange 432 of the penetration depth assembly 430 and the top surface 434 of the needle-penetration assembly 400 can be increased or decreased, thereby adjusting the penetration depth of the needle 410. It should be appreciated that the needle-penetration assembly 400 may, additionally or alternatively, include various other gears and levers to adjust the needle-penetration depth.

In some embodiments, in addition to the needle 410 and the penetration depth assembly 430, the needle-penetration assembly 400 can also include one or more stimulation generators 104 and controls. In such embodiments, the stimulation generator(s) 104 and controls can be integrally provided within the needle-penetration assembly 400. For example, in some embodiments, the needle-penetration assembly 400 can include a vibrating motor 440, a heat generator 442, and electronic controls 444. Other stimuli such as cold packs, electric or static shock generators, and massage systems can also be integrated into the needle-penetration assembly 400.

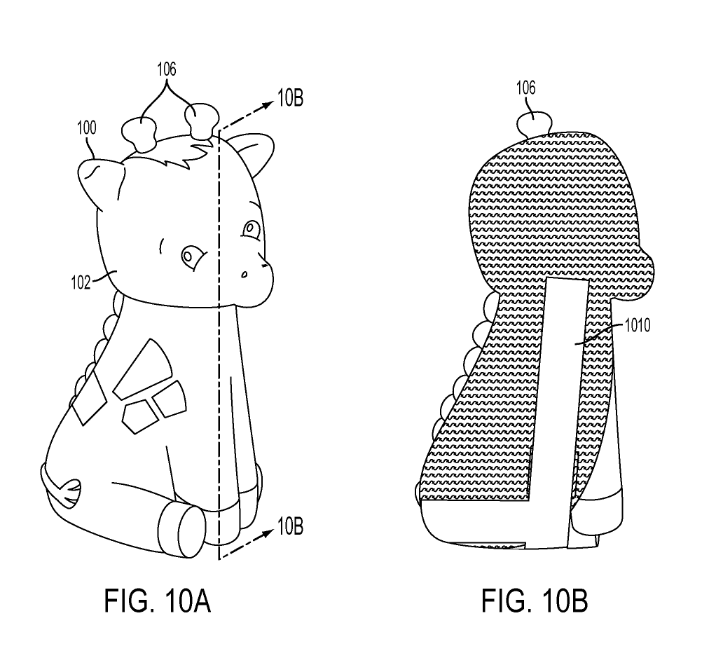

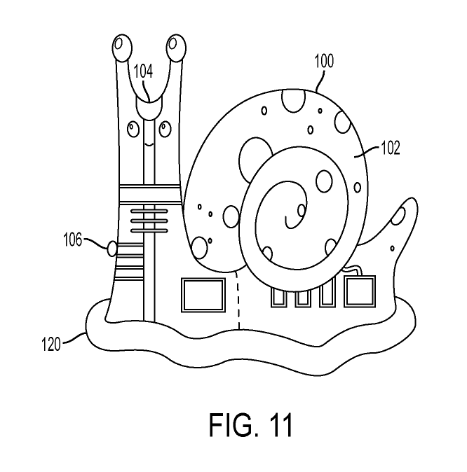

It should be appreciated that the encasement apparatus 100 can be provided in a large variety of shapes. Selections of some non-limiting examples are depicted in FIGS. 10-14. FIG. 10A-10B, for example, depicts a giraffe-like shape while FIGS. 11 and 12 depict a snail and imaginary character, respectively. As depicted in FIGS. 13 and 14, the encasement apparatus 100 can also be provided in non-threatening and fun shapes. As discussed below, in some embodiments, the needle penetration assembly 150 and/or the needle-penetration assembly 400 can be integrally installed into the encasement apparatuses 100 depicted in FIGS. 11-14.

Referring now to FIGS. 10A-10B, in some embodiments, the encasement apparatus 100 may include a housing 102 having an exterior shape that resembles a giraffe, as discussed. In such embodiments, the housing 102 can define an interior chamber 1010 configured to receive the needle-penetration assembly 400 illustratively shown in FIG. 9. In some embodiments, the encasement apparatus 100 includes an actuator 106. Interaction with the actuator 106 may cause the needle 410 of the needle-penetration assembly 400 to puncture the skin of a user. Additionally or alternatively, interaction with the actuator 106 may cause one or more stimuli to be generated by stimulation generator(s) 104 integral to the encasement apparatus.

Referring now to FIG. 11, in some embodiments, the encasement apparatus 100 may include a housing 102 having an exterior shape that resembles a snail, as discussed. In such embodiments, the housing 102 can define an interior chamber (not shown) configured to receive the needle-penetration assembly 400 illustratively shown in FIG. 9. The encasement apparatus 100 and/or the housing 102 can include on or more stimulation generators 104 for generating stimuli as discussed above. In some embodiments, the encasement apparatus 100 includes an actuator 106, which can be configured to cause the needle 410 of the needle-penetration assembly 400 to puncture the skin of a user and/or cause one or more stimulation generators 104 of the encasement apparatus 100 to generate stimuli. In some embodiments, the encasement apparatus 100 includes a base 120 configured to support the housing 102 and/or assist an operator in correctly aligning and placing the encasement apparatus 100 on a patient or user. As discussed, the base 120 may include a textured surface (not shown) and/or one or more stimulation generators 104.

Referring now to FIG. 12, in some embodiments, the encasement apparatus 100 may include a housing 102 having an exterior shape that resembles an imaginary character, as discussed. In such embodiments, the housing 102 can define an interior chamber (not shown) configured to receive the needle-penetration assembly 400 illustratively shown in FIG. 9. The encasement apparatus 100 and/or the housing 102 can include on or more stimulation generators 104 for generating stimuli as discussed above. For example, in some embodiments, the encasement apparatus 100 may include a stimulation generator 104 embodied as a speaker 160 for generating auditory stimuli. Additionally or alternatively, the encasement apparatus 100 may include a stimulation generator 104 embodied as a display 162 for generating visual stimuli. The encasement apparatus 100 may also include one or more stimulation generators 104 embodied as LEDs for generating additional visual stimuli. In some embodiments, the encasement apparatus 100 includes an actuator, which can be configured to cause the needle 410 of the needle-penetration assembly 400 to puncture the skin of a user and/or cause one or more stimulation generators 104 of the encasement apparatus 100 to generate stimuli.

As depicted in FIGS. 13 and 14, the encasement apparatus 100 can also be provided in non-threatening and fun shapes. For example, in some embodiments such as the one illustratively shown in FIG. 13, the encasement apparatus 100 may be embodied as the encasement apparatus 1302. The encasement apparatus 1302 may include some or all of the features of the encasement apparatus 100 in some embodiments. As illustratively shown, the encasement apparatus 1302 can have an exterior shape that is fun and pleasing to a user. Additionally, the encasement apparatus 1302 can have a housing 1304. The housing 1304 can include one or more stimulation generators for generating stimuli. For example, as illustratively shown, the encasement apparatus 1302 includes one or more LEDs 1306 to generate visual stimuli and a speaker 160 to generate audio stimuli.

In other embodiments such as the one illustratively shown in FIG. 14, the encasement apparatus 100 may be embodied as the encasement apparatus 1400. The encasement apparatus 1400 may include some or all of the features of the encasement apparatus 100 in some embodiments. As illustratively shown, the encasement apparatus 1400 can have an exterior shape that is fun and pleasing to a user. Additionally, the encasement apparatus 1400 can have a housing 1402. The housing 1402 can include one or more stimulation generators for generating stimuli. For example, as illustratively shown, the encasement apparatus 1400 includes one or more internal motors 1406 configured to generate vibrations and a vacuum and/or diaphragm 1404 to generate suction.

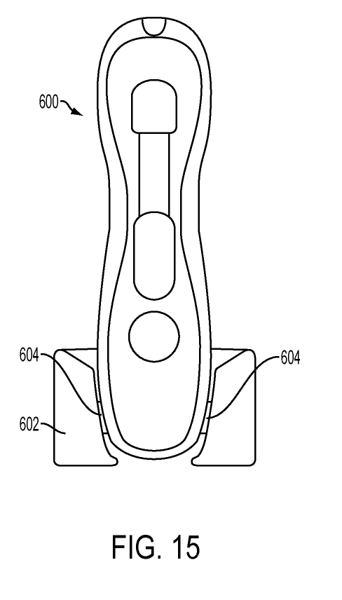

FIG. 15 illustrates an alternate embodiment of a needle penetration assembly 600 that can be associated with a portable stimulation assembly 602, where the stimulation assembly can be activated upon insertion of the needle penetration assembly 600 such that one or a plurality of actuators 604 are depressed. The stimuli, such as a vibration stimulus, can be activated upon depression of the actuators 604. It will be appreciated that the portable stimulation assembly 602 can be sized for placement in a pocket, kit, or other convenient location.

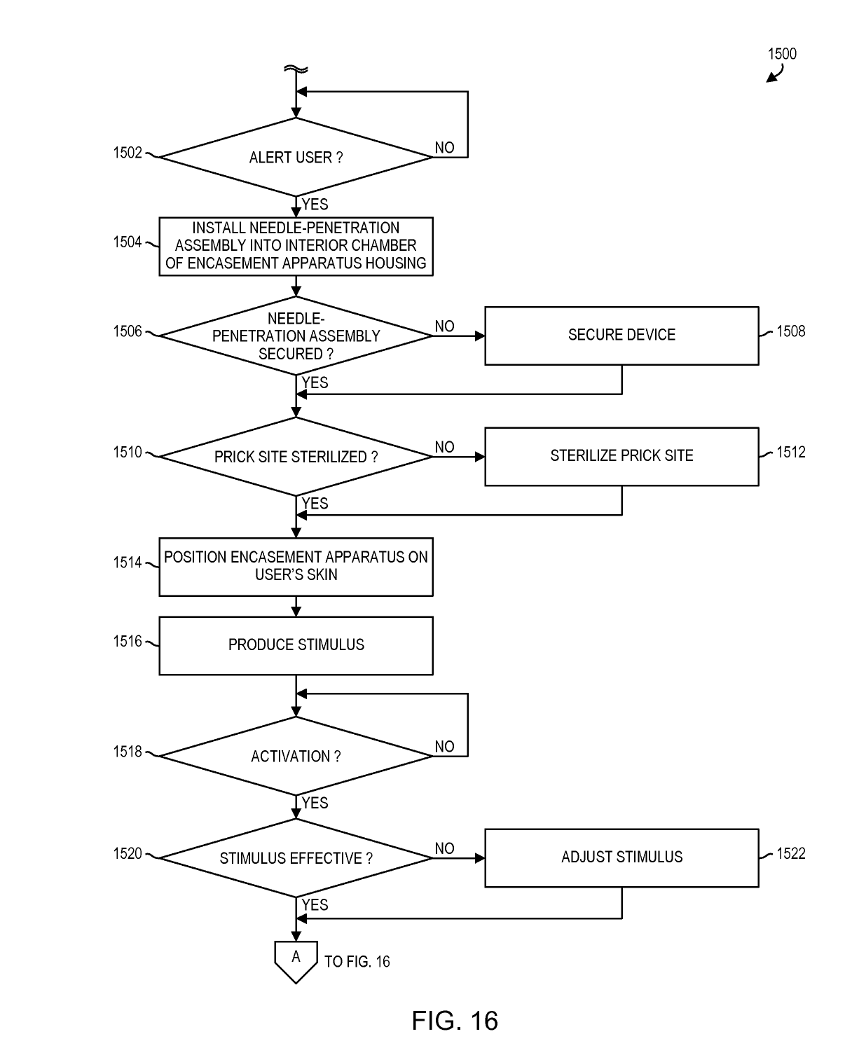

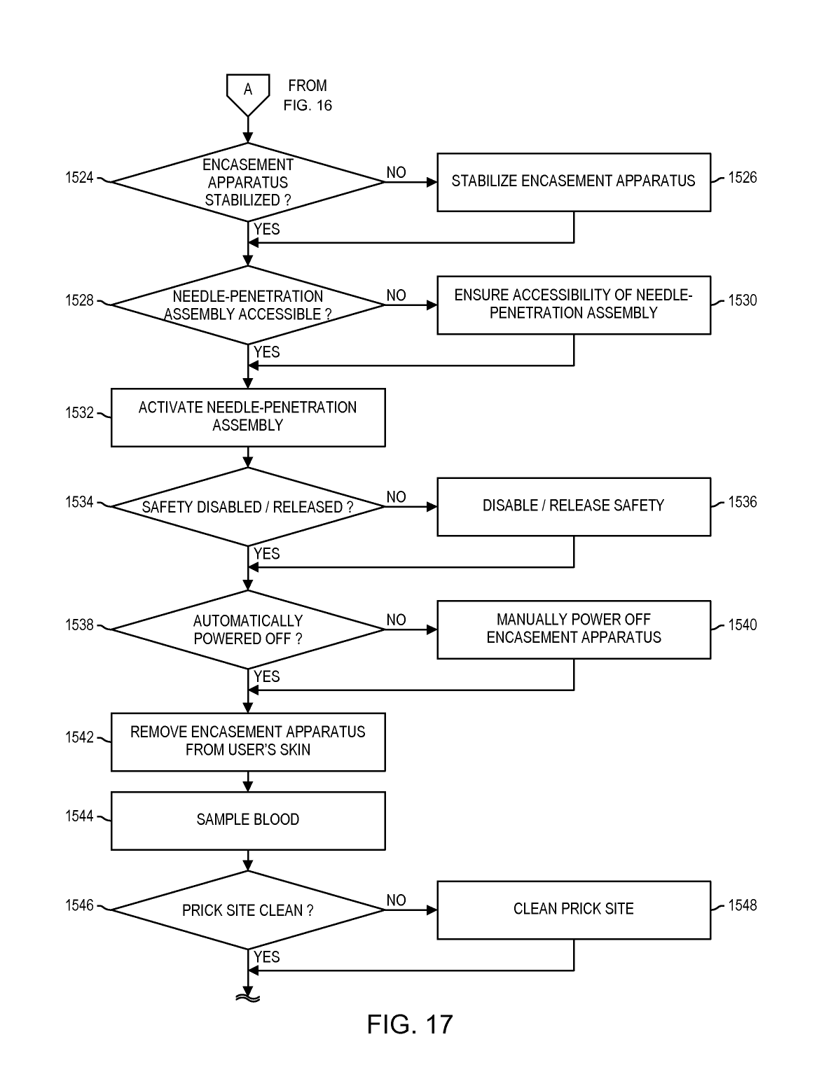

Referring now to FIGS. 16 and 17, at least one embodiment of a method 1500 for operating the encasement apparatus 100 begins with decision block 1502. In decision block 1502, it can be determined whether to alert or notify a user of the encasement apparatus 100. To do so, in some embodiments, a reminder or notification message may be transmitted to a mobile app and/or directly to a mobile computing device via a text message, email message, or any other type of message. The encasement apparatus can include a transmitter, receiver, controller, microcontroller, or any other suitable features to communicate remotely with a network such as the internet or a local area network, another encasement apparatus, a physician, or the like. In such embodiments, in response to receiving a reminder or notification message, the mobile computing device may be configured to vibrate, generate an audible sound, and/or display a corresponding message to the user. Additionally or alternatively, the encasement apparatus 100 can be configured to vibrate and/or generate an audible sound. The audible sound generated by the encasement apparatus 100 may be embodied as an escalating sound. In some embodiments, one of a plurality of reminders may be selected by the user via one or more operating modes of the encasement apparatus 100. Additionally or alternatively, in some embodiments, the encasement apparatus 100 can be configured to vibrate and/or generate audible sounds based on a customizable timer. In such embodiments, the user may select or customize an operating mode, a reminder, and/or a time via interactions with the encasement apparatus 100 or through interactions with the mobile computing device, which may be configured to transmit the user's selections or customizations to the encasement apparatus 100. It should be appreciated that alerting or reminding the user may encourage treatment or monitoring compliance. Referring back to decision block 1502, if it is determined that the user is to be alerted, the method 1500 advances to block 1504. If, however, it is determined instead that the user is not to be alerted, the method 1500 loops back to block 1502 and monitoring for a user alert event continues.

In block 1504, the needle-penetration assembly 150 is installed and/or mounted into the interior chamber 110 of the housing 102 of the encasement apparatus 100. It should be appreciated that in embodiments in which the needle-penetration assembly 400 is used, the needle-penetration assembly 400 may instead be installed into a corresponding interior chamber, such as the interior chamber 1010 illustratively shown in FIG. 10B. However, for clarity of the description, the needle-penetration assembly 150 and the interior chamber 110 will be referred to for the remaining discussion of the method 1500.

In decision block 1506, it is determined whether the needle-penetration assembly 150 is securely installed and/or mounted in the interior chamber 110. If, in decision block 1506, it is determined that the needle-penetration assembly 150 is securely installed and/or mounted in the interior chamber 110 of the encasement apparatus 100, the method 1500 advances to decision block 1510. If, however, it is determined instead that the needle-penetration assembly 150 is not securely installed and/or mounted in the interior chamber 110, the method 1500 advances to block 1508.

In block 1508, one or more straps of the encasement apparatus 100 and/or the interior chamber 110 may be tightened to facilitate securing and/or mounting the needle-penetration assembly 150 into the interior chamber 110. Additionally or alternatively, the needle-penetration assembly 150 can be slid along one or more grooves of the interior chamber 110 for secure alignment. In some embodiments, the needle-penetration assembly 150 can be placed in contact with a gripping conformation configured to hold the needle-penetration assembly 150 into place. The needle-penetration assembly 150 can also be placed in contact with a material integral to the interior chamber 110 that includes an adhesive to prevent slippage. In some embodiments, a hinge or swinging door of the interior chamber 110 and/or the encasement apparatus 100 may hold the needle-penetration assembly 150 into place. Additional assembled components of the encasement apparatus 100 and/or the interior assembly may also provide support and/or secure the needle-penetration assembly 150 into place.

In decision block 1510, it is determined whether the prick site on the skin of the patient is sterile. In some embodiments, it can also be determined whether the prick site is a suitable prick site. For example, in some embodiments, it is detected whether the prick site area on the skin of the patient is a previous prick site, shows evidence of skin disease, exhibits excessive grease, exhibits excessive dirt, exhibits excessive hair, exhibits calluses, and/or shows evidence of lipoma. If, in decision block 1510, it is determined that the prick site is sterile, the method 1500 advances to block 1514. If, however, it is determined instead that the prick site is not sterile, the method 1500 advances to block 1512. In block 1512, the prick site on the skin of the patient is sterilized. To do so, in some embodiments, the area of skin corresponding to the prick site may be swiped or sprayed with a suitable disinfecting and/or sterilization liquid or solution to prevent contamination. detection of non-suitable sites such as previous prick sites, skin disease, excessive grease, excessive dirt, excessive hair, calluses, lipoma, and/or skin diseases

In block 1514, the encasement apparatus 100 can be positioned on the skin of the patient. In some embodiments, the encasement apparatus 100 includes sensory and/or physical orientation indicators to facilitate proper positioning of the apparatus. Additionally or alternatively, the encasement apparatus 100 can include one or more sensors configured to detect when the encasement apparatus 100 is ready for use. In some embodiments, the encasement apparatus 100 includes one or more safety mechanisms configured to automatically or manually disengage when the encasement apparatus 100 is ready for use. The encasement apparatus 100 may also include one or more adjustable contact-pressure modifiers. In some embodiments, the encasement apparatus includes one or more sensory indicators configured to provide the user with instructions on how to operate the encasement apparatus 100.

In block 1516, one or more of the stimulation generators 104 of the encasement apparatus 100 generate or produce auditory, visual, somatosensory, gustatory, and/or electric stimuli. For example, in some embodiments, the stimulation generator(s) 104 generate cold and/or hot stimuli. In other examples, the stimulation generator(s) 104 generate steady, periodic, and/or random vibrational stimuli. For instance, the stimulation generator(s) 104 may generate continuous mechanical stimuli such as ramping vibration. Sensory distractions such as visual, physical, olfactory, auditory, and/or gustatory stimuli may also be generated by the stimulation generator(s) 104. For example, the stimulation generator(s) 104 generate may generate audible signals such as a countdown, visual feedback such as flashing lights, pleasant scents, and/or positive psychological feedback such as cheering and/or compliments. Additionally, one or more of the stimulation generator(s) 104 may apply electro static stimuli, magnetic stimuli, quantum mechanical interactions, psychological stimuli, and/or pressure stimuli to the skin of the user. Additionally, in some embodiments, one or more of the stimulation generators 104 may project an image onto a surface and/or as a holographic display.

In decision block 1518, it is determined whether the stimuli was activated and presented to the user. If, in decision block 1518, it is determined that the stimuli was activated and presented to the user, the method 1500 advances to decision block 1520. If, however, it is determined instead that the stimuli was not activated or not presented to the user, the method 1500 loops back to decision block 1518 and monitoring for activation continues.

In decision block 1520, it is determined whether the stimuli were effective. That is, it is determined whether or not the stimuli had their intended effect on the patient. For example, in some embodiments, the encasement apparatus 100 may include one or more perspiration meets and/or heat rate monitors to detect user discomfort. If, in decision block 1520, it is determined that the stimuli were effective, the method 1500 advances to decision block 1524. If, however, it is determined instead that the stimuli were not effective, the method 1500 advances to block 1522.

In block 1522, the stimuli generated by the stimulation generators 104 of the encasement apparatus 100 are adjusted. For example, in some embodiments, buttons and/or controls of the encasement apparatus 100 and/or the needle-penetration assembly 150 are used to manually adjust the stimuli intensity. In other embodiments, the stimuli are adjusted automatically. For example, the stimuli generated by the stimulation generators 104 may be automatically adjusted in response to environmental feedback such as the temperature. In another example, the stimuli generated by the stimulation generators 104 may be automatically adjusted based on user preferences such as prior setting by the user. Additionally, the stimuli generated by the stimulation generators 104 may be adjusted in response to a voice activated command received by the encasement apparatus 100. In some embodiments, the voice activated commands can be transmitted by a mobile computing device of the user.

In decision block 1524, it is determined whether the encasement apparatus 100 is stabilized relative to the user. For example, in some embodiments, the encasement apparatus 100 (or a component thereof) may include an accelerometer configured to detect whether the encasement apparatus 100 is stabilized. Additionally or alternatively, in some embodiments, the encasement apparatus 100 may include indicators configured to facilitate alignment and stabilization of the encasement apparatus 100. If, in decision block 1524 it is determined that the encasement apparatus 100 is stabilized relative to the user, the method 1500 advances to decision block 1528. If, however, it is determined instead that the encasement apparatus 100 is not stabilized relative to the user, the method 1500 advances to block 1526.

In block 1526, the encasement apparatus 100 is stabilized to hold the encasement apparatus 100 in place. To do so, in some embodiments, the encasement apparatus 100 is held firmly on the skin of the user. Additionally or alternatively, in some embodiments, the encasement apparatus 100 may be strapped in place to position the encasement apparatus 100. In some embodiments, compression (e.g., via clamps, etc.) and/or adhesives are applied to stabilize and hold the encasement apparatus 100 in place. The encasement apparatus 100 can also be gripped for stabilization and placement. In some embodiments, a suctioning force from the encasement apparatus 100 may be applied to the skin surface of the user to stabilize and hold the encasement apparatus 100 in place.

In decision block 1528, it is determined whether the needle-penetration assembly 150 is accessible. If, in decision block 1528 it is determined that the needle-penetration assembly 150 is accessible, the method 1500 advances to block 1532. If, however, it is determined instead that the needle-penetration assembly 150 is not accessible, the method 1500 advances to block 1530.

In block 1530, accessibility of the needle-penetration assembly 150 is ensured. To do so, in some embodiments, the needle-penetration assembly 150 may be accessed and/or repositioned via the aperture 132 of the housing 102. In some embodiments, the needle-penetration assembly 150 may be repositioned within the interior chamber 110 to provide access to settings, buttons, and/or controls of the needle-penetration assembly 150.

In block 1532, the needle-penetration assembly 150 and/or the encasement apparatus 100 is activated or actuated. In some embodiments, the needle-penetration assembly 150 and/or the encasement apparatus 100 can be activated via the actuator 106. Additionally or alternatively, the needle-penetration assembly 150 and/or the encasement apparatus 100 can be activated via one or more buttons and/or controls of the needle-penetration assembly 150.

In decision block 1534, it is determined whether safety mechanisms of the needle-penetration assembly 150 and/or encasement apparatus 100 are disabled. If, in decision block 1534, it is determined that the safety mechanisms of the needle-penetration assembly 150 and/or encasement apparatus 100 are disabled, the needle 410 of the needle-penetration assembly 150 is released and the method 1500 advances to decision block 1538. If, however, it is determined instead that one or more safety mechanisms of the needle-penetration assembly 150 and/or the encasement apparatus 100 are not disabled, the method 1500 advances to block 1536.

In block 1536 one or more safety mechanisms of the needle-penetration assembly 150 and/or encasement apparatus 100 are disabled or deactivated. For example, in some embodiments, a locking or unlocking mechanism such as a latch and/or shutter can be operated or interfaced with to disable a corresponding safety mechanism. Additionally, in some embodiments, user recognition components of the encasement apparatus 100 and/or a mobile computing device can be used to activate or deactivate one or more of the safety mechanisms.

In decision block 1538, it is determined whether the encasement apparatus 100 and/or the needle penetration assembly 150 is automatically turned off after use (e.g., after actuation/activation of the needle 410). In some embodiments, the encasement apparatus 100 and/or the needle penetration assembly 150 can automatically be turned off in response to a timer, detecting a loss of contact between the skin of the user and the encasement apparatus 100, generating positive feedback to the user, and/or detecting that a safety mode of the encasement apparatus 100 and/or the needle penetration assembly 150 has been reengaged. If, in decision block 1538, it is determined that the encasement apparatus 100 and/or the needle penetration assembly 150 are automatically turned off, the method 1500 advances to block 1542 in which the encasement apparatus 100 is removed from the skin of the user. If, however, it is determined instead that the encasement apparatus 100 and/or the needle penetration assembly 150 are not automatically turned off, the method 1500 advances to block 1540. In block 1540, the encasement apparatus 100 and/or the needle penetration assembly 150 are manually turned off.

In block 1542, the encasement apparatus 100 is removed from the skin of the user. In block 1544, blood from the prick site is sampled. In some embodiments, a meter such as the blood glucose meter 300 may sample the blood. In such embodiments, automatic mechanical suction can be used to deliver the blood sample into an analysis compartment (e.g., the test strip 303, testing sensor, or testing chamber) of the blood glucose meter 300 (or other testing meter). It will be appreciated that chambers can be used for storage, disposal of used products, be SHARPS containers, and can automatically accept, dispense, or expel one or a plurality of items when an actuator is actuated. For example, in one version, a sticker can automatically be expelled from the encasement device when the actuator is depressed to prick the skin of the patient.

In decision block 1546, it is determined whether the prick site on the skin of the patient is clean. If, in decision block 1546, it is determined that the prick site is clean, the method 1500 completes. If, however, it is determined instead that the prick site is not clean, the method 1500 advances to block 1548. In block 1548, the prick site on the skin of the patient is cleaned. To do so, in some embodiments, the area of skin corresponding to the prick site may be swiped or sprayed with a suitable disinfecting and/or sterilization liquid or solution to clean the area of contaminants or blood. In some embodiments, device-to-skin contact may be controlled to minimize blood smears and facilitate cleaning of the prick site. Additionally, blood flow may be constricted to prevent further bleeding and facilitate cleaning of the prick site.

In embodiments in which a mobile computing device is used in conjunction with the encasement apparatus 100, the mobile computing device may be embodied as any type of computing device capable of performing the functions described herein. As such, the mobile computing device may include devices and structures commonly found in computing devices such as processors, memory devices, communication circuitry, and data storages, which are not shown in the figures for clarity of the description. In some embodiments, the mobile computing device is configured to send and receive user selections and/or customizations to and from the encasement apparatus 100. For example, in some embodiments, the mobile computing device may receive one or more reminder messages sent by the encasement apparatus. Additionally or alternatively, the mobile computing device may transmit user selections and/or customization relating to, among others, operating modes, reminder settings, and timer settings of the encasement apparatus 100.

In various embodiments disclosed herein, a single component can be replaced by multiple components and multiple components can be replaced by a single component to perform a given function or functions. Except where such substitution would not be operative, such substitution is within the intended scope of the embodiments.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto.

We claim:

1. A needle-penetration pain management system for managing needle penetration pain, the needle-penetration pain management system comprising:
(a) a needle-penetration assembly including a needle, the needle having a proximal end and a distal end;
(b) an encasement apparatus including;
(i) a housing, the housing having a front surface, a rear surface, and a base, wherein the housing at least partially defines an interior chamber for installing the needle-penetration assembly at least partially therein,
(ii) a stimulation generator for producing stimuli, the stimulation generator being coupled with the housing, and
(iii) an actuator in communication with the needle-penetration assembly for causing the needle to puncture a skin surface of a user in response to actuation.

2. The needle-penetration pain management system of claim 1, further comprising an attachment mechanism such that the encasement apparatus is selectively coupled with the user.

3. The needle-penetration pain management system of claim 1, further comprising a suction system.

4. The needle-penetration pain management system of claim 1, wherein the stimuli includes vibration.

5. The needle-penetration pain management system of claim 1, wherein the stimuli is vibration in combination with a stimulus selected from the group consisting of a visual stimulus, an aural stimulus, a temperature stimulus, a texture stimulus, a physical stimulus, a chemical stimulus, a somatosensory stimulus, a gustatory stimulus, and combinations thereof.

6. The needle-penetration pain management system of claim 1, wherein actuating the actuator causes the stimuli to be activated and the needle to be actuated substantially simultaneously.

7. The needle-penetration pain management system of claim 1, wherein the encasement apparatus is configured to accept and dispense a plurality of items upon actuation of the actuator.

8. The needle-penetration pain management system of claim 1, wherein the stimulation generator resembles an anatomical structure of an organism.

9. The needle-penetration pain management system of claim 1, wherein the needle penetration assembly is a lancing system.

10. The needle-penetration pain management system of claim 1, wherein the housing defines at least one chamber for storage or disposal of materials.

11. The needle-penetration pain management system of claim 1, wherein the encasement apparatus is configured to accept a plurality of off-the-shelf needle penetration assemblies.

12. The needle-penetration pain management system of claim 1, wherein the housing is shaped to resemble an organism, object, or structure.

13. The needle-penetration pain management system of claim 1, wherein the stimuli is at least one of a steady vibrational stimulus, a periodic vibrational stimulus, or a random vibrational stimulus.

14. The needle-penetration pain management system of claim 1, wherein the housing defines an aperture in the base of the encasement apparatus for insertion of the needle penetration assembly.

15. The needle-penetration pain management system of claim 1, further comprising a plurality of stimulation generators.

16. The needle-penetration pain management system of claim 1, further comprising a controller, a transmitter, and a receiver associated with the housing such that the encasement assembly is configured to communicate with a remote electronic device via a network.

17. The needle-penetration pain management system of claim 1, wherein the remote electronic device is a mobile smartphone.

18. A needle-penetration pain management system for managing needle penetration pain, the needle-penetration pain management system comprising:
(a) a needle-penetration assembly including a needle, the needle having a proximal end and a distal end;
(b) an encasement apparatus including;
(i) a housing resembling an animal, the housing having a front surface, a rear surface, and a base, wherein the housing at least partially defines an interior chamber and an aperture in the base for installing the needle-penetration assembly at least partially therein,
(ii) a first chamber, defined by the housing, for the storage of materials;
(iii) a first stimulation generator for producing a vibrational stimulus, the first stimulation generator being coupled with the housing, wherein the first stimulation generator is selectively adjustable between a plurality of modes of vibrational stimuli,
(iv) a second stimulation generator for producing a visual stimulus;
(v) a third stimulation generator for producing an audible stimulus;
(vi) a transmitter, a receiver, and a controller associated with the housing, wherein the transmitter and the receiver are configured to communicate with a remote device over a network; and
(vii) an actuator in communication with the needle-penetration assembly for causing the needle to puncture a skin surface of a user in response to actuation.

19. The needle-penetration pain management system of claim 18, wherein the remote device is a mobile smartphone.

20. A needle-penetration pain management system comprising:
(a) a needle assembly means;
(b) an encasement assembly means having;
(i) a housing means,
(ii) a stimulation means, and
(iii) an actuator means.
